(19) United States
(12) Patent Application Publication
(10) Pub. No.: US 2013/0185088 A1
(43) Pub. Date: Jul. 18, 2013

(54) SYSTEM AND METHOD FOR MANAGING MEDICAL CARE USING RFID AND BIOMETRIC AUTHENTICATION TECHNOLOGIES

(71) Applicant: BRIAN JEFFRY BRYANT, Ft. Worth, TX (US)

(72) Inventor: BRIAN JEFFRY BRYANT, Ft. Worth, TX (US)

(21) Appl. No.: 13/349,175

(22) Filed: Jan. 12, 2012

(51) Int. Cl. G06Q 50/22

(52) U.S. Cl. 705/2

(57) ABSTRACT

A medical management system and method operates over a computer network to supervise and manage services and medication provided by a care provider to one or more patients in a care facility. The system uses RFID and biometric authentication technologies to determine appropriate proximity of the patient to the care provider and the identities of the patient and care provider, respectively, both before and after a service event to effectively manage and document interactions between the care provider and the patient. Electronic voice technology and electronic notification generation capability are also incorporated to audibly confirm identities and services, and to notify supervisory personnel of anomalies in service events, respectively.

### BACKGROUND OF THE INVENTION

1. Field of the Invention

The present invention is related to the field of medical monitoring systems and methods and, more particularly, to a medical care management system and method using biometric and RFID technology to enable participation in and to verify the completion of prescribed care events, and to verify that services are provided in appropriate locations and at appropriate times with respect to all authorized participants.

2. Description of the Related Art

There are many challenges associated with managing the care of patients/residents within assisted living and/or other health care related facilities and environments where medications are dispensed and various services are managed by staff members. One of these challenges is to ensure that interactions between the staff and the residents of such facilities are supervised and that care events are properly completed and documented. In care facility environments, the patient may resent being there or for some other reason may resist submitting to various care events including the taking of medications. Care providers face difficulties in trying to convince the patient to cooperate. Depending upon the personalities of the two parties and other factors, the care provider's efforts to force the patient into complying can lead to greater resistance on the part of the patient. In some cases, the care provider may become frustrated and simply document that the care event was completed when, in fact, it was not. In such a case, the care provider may dispose of the medication, resulting in loss to both the patient's standard of care and the care facility's inventory. In other cases, when questioned by a supervisor or visiting family member as to why the patient is not accepting medication, the patient may deny having been offered the medication. Careful tracking and control of medical inventory distribution and the administration of services can significantly reduce the possibility of human error and also mitigate the fraud, waste and abuse that are sometimes prevalent in the health care industry.

Therefore, a need exists for a system and method of managing the pertinent aspects of providing of care; resident services, medication, dietary and finances, and operations of assisted living facilities and other health care organizations with greater efficiency, accountability, reliability and risk mitigation.

### SUMMARY OF THE INVENTION

Accordingly, the present invention is directed to a computerized system and method of real-time management and tracking of patient/resident care services that provide verification that the services were provided and completed in connection with specific patients/residents (hereinafter referred to generally as “patients”) and by a specific care provider at a specified time and location. As used herein, services are often referred to as “care events”; the complete process by which a care provider and a patient complete an individual care event, including pre and post authentication, is called a transaction.

The system uses RFID technology including RFID tags to verify that the location of all participants in the care event is appropriate for a given proximity-based service. Once the location has been verified, both the patient and the care provider biometrically authenticate their respective identities. If the patient is unable or unwilling to authenticate his or her identity, a second employee authenticates his or her identity and acknowledgment of the transaction as a witness. Any anomalies in this process will generate an immediate notification to supervisors. Following successful completion of the proximity-based biometric verification of the identities of both the patient and the care provider, or of the care provider and a witness, the scheduled service(s) is/are performed. When all services have been completed, the PCU makes a final verification of the location by accessing the appropriate RFID tag again and then the patient and the care provider, or the care provider and the witness, again biometrically authenticate their identities. Requiring a second biometric authentication at the end of the transaction serves to positively verify and also time stamp completion of the service transaction. Time stamping serves to document when a transaction was started and completed and allows supervisors to compare the length of time taken to complete the transaction with “typical” time periods taken when performing other transactions of the same type. In addition, the persons authenticating their identities at the completion of a care event should be the same as the persons who authenticated their identities prior to the care event. If, for some reason, one of the care providers is required to leave the transaction prior to completion, another authorized person must be available to take over. The care provider who is leaving must biometrically authenticate that he/she is departing, the new care provider must biometrically authenticate himself or herself in order to join the transaction, and supervisors are notified of the activity. By requiring that only authorized care providers start, complete and document a given care event, a chain of accountability is ensured for each transaction.

The present invention utilizes a portable care unit including a computer having a touch screen display and an associated biometric scanning device for completing the biometric authentication. The computer also includes an electronic voice module that generates a virtual persona (VP) which acts as a vocal participant in the patient/care provider transaction. The virtual persona, which is preferably incorporated within the computer, audibly confirms the identity of the patient and then provides the patient with a description of the service(s) to be performed. For hearing-impaired participants in a transaction, the VP provides a visual display of the appropriate information on the computer's touch screen. As used herein, “visual display” is intended to include, but is not limited to, text, graphics, photo images, etc. In the event that the patient refuses the service, the virtual persona audibly and/or visually confirms the patient's refusal and informs the patient of their non-compliance with the plan of care established by the patient's physician. The participation of the computer-generated VP, particularly when in audible mode, serves to provide a virtual third party who is neutral to the transaction. In the event of a disagreement between the care provider and the patient, the “mediation” provided by the audible comments of the VP can often persuade the patient to cooperate and acquiesce to the care event. Hence, the participation of the VP not only results in a greater number of successfully completed transactions, but also takes the burden of the patient's lack of cooperation off of the care provider as the VP becomes the authority figure.

The present invention further includes an electronic notification capability that serves to inform supervisory personnel or other remote parties when anomalies or failures to adhere to an established sequential plan of care have occurred. For example, if the patient has refused a service, the system electronically generates and transmits a secure message to supervisory personnel for their review and action. Similarly, if the service is a particular test and, upon completion of the test, the results are outside of an accepted range, the system electronically generates and transmits a notification message to appropriate supervisory personnel or other remote/interested parties for immediate review and necessary action.

In view of the foregoing, it is an object of the present invention to provide an automated and computer-based system and method for managing the care, medication, and operations of assisted living facilities and other facilities providing health care services that reduce the possibility of human error and mitigate fraud, waste and abuse through the use of biometrics and RFID authentication for all two-party transactions undertaken within such facilities.

Another object of the present invention is to provide a health care management system and method in accordance with the preceding object that effectively manage interactions between care providers and patients by providing computer-directed instructions as to the service to be performed, and when and where the service is to be performed, and by providing electronic documentation of the identity of the specific patient and care provider involved in the service, and whether in fact the service was performed.

Yet another object of the present invention is to provide a health care management system and method in accordance with the preceding objects that make innovative use of electronic voice technology to generate a virtual persona that personalizes the delivery of services while also audibly and/or visually supervising all interactions between the care provider and the patient in order to mitigate possible misunderstandings and/or conflicts between the care provider and the patient.

Still another object of the present invention is to provide a health care management system and method in accordance with the preceding objects that use a network connected application with a state-of-the-art user interface on a touch-screen device, with the two-party biometric authentication being performed using Vascular Infrared Verification (VIV) technology.

A further object of the present invention is to provide a health care management system and method in accordance with the preceding objects that incorporate RFID technology in which tags are used to verify that the location of a proposed care event is appropriate and, if verification is successful, to initiate the start of a timed period within which biometric authentication must occur before a care event is allowed to commence.

Another object of the present invention is to provide a health care management system and method in accordance with the preceding objects that biometrically authenticate the identity of the patient and an authorized care provider both before and after completing a service transaction.

Still another object of the present invention is to provide a health care management system and method in accordance with the preceding objects that is programmed to function according to a pre-determined schedule of events at specified locations such that a specific care event can only take place within a scheduled time window at a scheduled location, reducing the risk of human error and mitigating fraud and other forms of health care system abuse.

A yet further object of the present invention is to provide a health care management system and method in accordance with the preceding objects that suspend an attempted transaction and automatically generate a real-time notification to supervising personnel whenever an anomaly or action occurs that is outside the scheduled time frame or acceptable results range, or which indicates non-compliance by the patient and/or care provider with required system procedures.

These together with other objects and advantages which will become subsequently apparent reside in the details of construction and operation as more fully hereinafter described and claimed, reference being made to the accompanying drawings forming a part hereof, wherein like numerals refer to like parts throughout.

### BRIEF DESCRIPTION OF THE DRAWINGS

FIG. 1 is a block diagram of the medical care management system as implemented using a computer network in accordance with the present invention.

FIG. 2 is a block diagram of the medical care management system of FIG. 1 and further including a distributed computer network such as the Internet.

FIGS. 3A and 3B are a flow chart of the method steps of using the medical management system shown in FIG. 1 in conducting a representative test by which the measuring of blood sugar is tracked and documented, and the blood sugar level is used to determine the medicinal and dietary treatment as specified in the patent's plan of care, in accordance with the present invention.

FIGS. 4A, 4B, 4C and 4D are a flow chart of the method steps by which medications are dispensed to a patient in accordance with the present invention.

FIG. 5 is a flow chart of the regular review process undertaken by the virtual persona component of the present invention.

FIG. 6 is a flow chart of the transaction monitoring function performed by the virtual persona component of the present invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar purpose.

Accordingly, the present invention is directed to an RFID and Biometric Authentication (RABA) system designed to be a totally integrated hardware and software solution to the challenges of managing the care, dispensing of medication and operations of medical facilities such as assisted living facilities. The RABA system operates to verify and manage care events including medication dispensement relating to one or more persons as provided by a care provider. The care provider moves throughout a facility where the RABA system is implemented with a computerized cart or other portable device, generally referred to herein as a portable care unit (PCU), that is equipped with an RFID tag reader and an associated biometric scanning device. The PCU is also equipped with a display which is preferably a touch screen display for data entry.

Each person having their care events tracked by the RABA system has an associated fixed location RFID tag in their room or other designated area. According to a preferred embodiment, the RABA system determines the geographic location of the PCU in relationship with the tag using wireless location data acquired by the tag reader device as will be discussed hereinafter. The RABA system introduces increased efficiency, accountability, reliability and risk mitigation through the use of biometric and RFID technologies to authenticate all two-party transactions, thus effectively managing interaction between care providers and patients and documenting the location at which services were provided, recording the exact time that the services were provided and how long it took to provide the services, what services were provided and whether, in fact, the services were actually successfully administered.

The RABA system of the present invention utilizes the RFID tags placed in defined patient locations and the tag readers incorporated within the PCUs that accompany the care provider to verify the proximity of the care provider and the patient at a defined location prior to initiating a care event. According to a preferred embodiment, the RFID tags are also used to verify the proximity of the care provider and the patient again after the care event is complete. The identities of both the patient and the care provider are authenticated biometrically both before and after the care event. By requiring that both proximity and identity be verified to both start and complete a care event, and that only authorized care providers are present and responsible for any given transaction from start to completion, the RABA system provides an increased level of security in terms of ensuring that a care event or bundle of pre-determined care events are completed according to schedule and properly documented. In addition, completion of the first RFID tag verification starts a time period within which the necessary biometric authentications must be completed. This time period is set by the supervisor and may be on the order of about three minutes. As would be understood, however, the time period may be shorter or longer as considered necessary. If the biometric authentications are not completed within the established time frame, the system electronically notifies a supervisor and the care event cannot proceed without supervisor authorization.

As is known in the art, tag readers locate RFID tags through electromagnetic interrogation of a spatial area to determine the presence of an object or person. One such system is described in U.S. Pat. No. 7,839,266 to Hoglund et al., the disclosure of which is hereby expressly incorporated by reference as is fully set forth herein.

In general, the RFID tag is an electromagnetic antenna and/or signal receiver which responds either passively or actively to announce the presence (or absence) of the tag within a controlled region defined by a broadcasted electromagnetic interrogation signal. Preferably each RFID tag sends an identification signal containing information that identifies the RFID tag with respect to other RFID tags. The RFID tag may store this identification information and, optionally, other information in a memory, such as in a non-volatile EEPROM. None, some or all of the other information may be included in the identification signal.

An RFID tag may be attached to, or included or enclosed in, an item, including a product or a person, to facilitate automatic identification of the item.

The present invention may be implemented using either passive or active RFID tags. A passive RFID tag is an RFID tag that does not require an internal power source and includes a transponder that sends an identification signal in response to receiving an “interrogation” signal sent by a transmitter. When using a passive RFID tag, the care provider's PCU may include a transmitter that is operative to interrogate the passive RFID tag.

An active RFID tag is an RFID tag that has an associated or internal power source. An active RFID tag may be capable of sending an identification signal without being activated by a received interrogation signal, and the tag may transmit its identification signal continuously or intermittently.

In an alternate configuration, the identification transmitter may be embodied as a Bluetooth transmitter or another appropriate transmitter as would be known by persons skilled in the art. In other alternative embodiments, the RABA system could determine the geographic location of a care provider's PCU and fixed location tag using wireless location data acquired via a wireless network connection (e.g., “WiFi” connection point) or based on wired plug location and computer identification such as Ethernet or MAC address in conjunction with a predetermined map associating a wired plug location or address with a geographic location.

Whether passive or active RFID tags or other transmitters are used, the tags communicate with a reader incorporated within the PCU. The reader is a device having a receiver that is capable of receiving an identification signal from an identification transmitter. A reader may also include a transmitter for sending an interrogation signal as needed for passive RFID tags. If the reader includes such a transmitter, the reader may operate the transmitter continuously or intermittently.

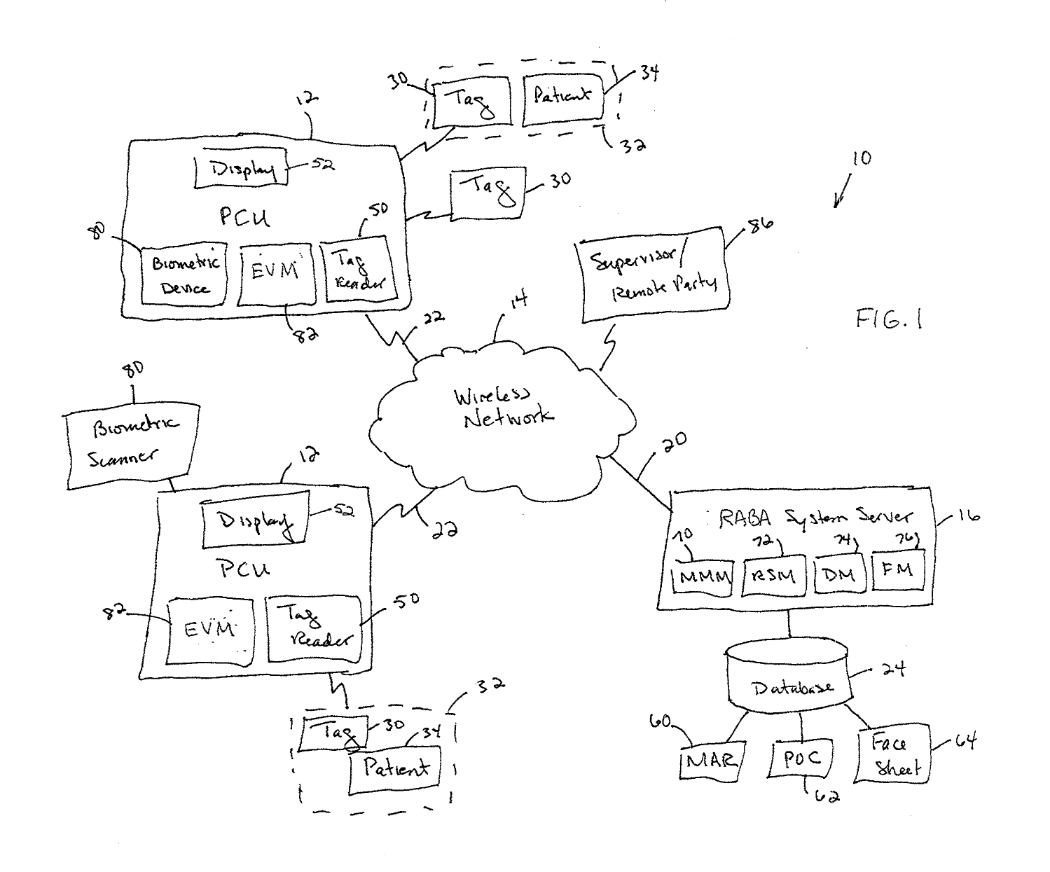

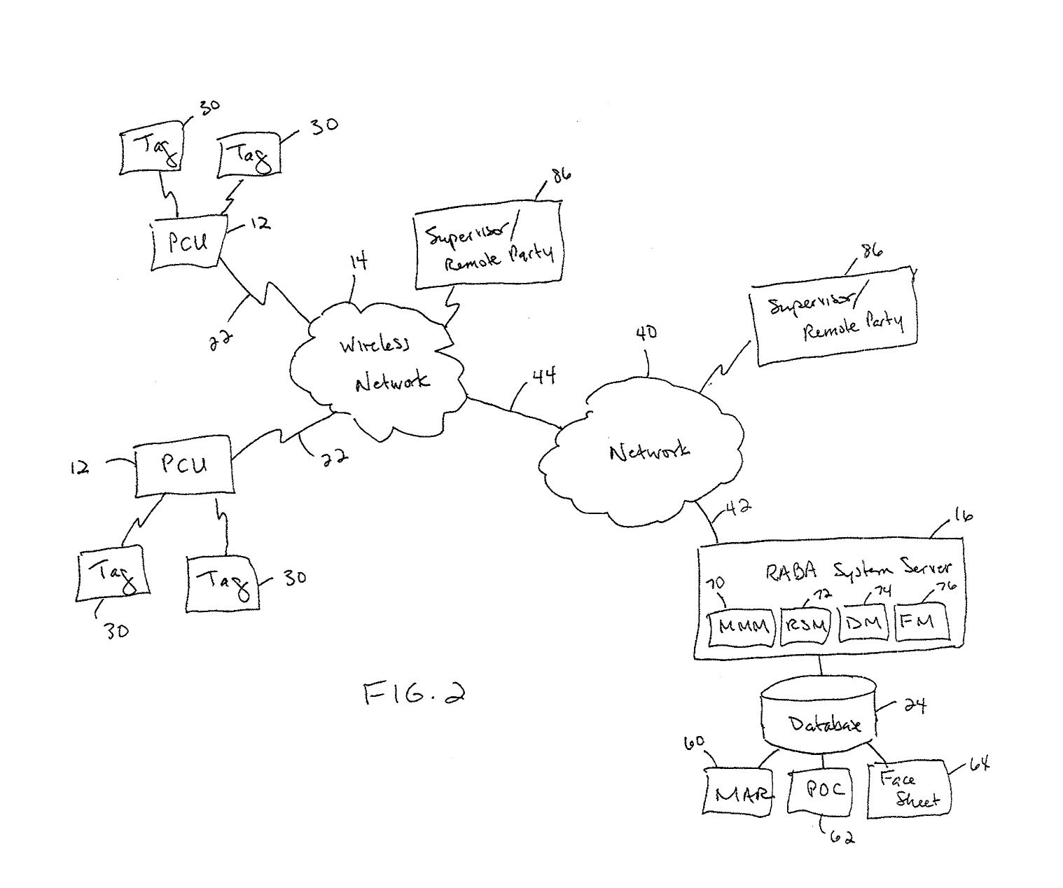

FIG. 1 is a block diagram of an exemplary context in which embodiments of the RABA system, generally designated by reference numeral 10, may be used. As noted, one or more care provider PCUs 12 may communicate, via a wireless computer network 14, with a RABA server 16. The wireless network 14 may be a data network such as a Short Message Service (SMS) network, a cellular network, a local wireless network (Bluetooth, Wi-Fi, etc.) or other wireless network. According to one preferred embodiment shown in FIG. 2, the wireless network 14 also couples to a network 40. In one embodiment, the network 40 includes at least a portion of the Internet. In another embodiment, the network 40 is a local area network or a wide area network. In general, the network 40 can be a wired network, a wireless network or both.

The RABA server 16 may be connected to the wireless network 14 via a wireless connection or, as shown in FIG. 1, via a wired connection 20. Alternatively, the server 16 may be connected to the network 40 by a wired connection 42 which is, in turn, connected to the wireless network 14 by a wired connection 44, as shown in FIG. 2. Turning back to FIG. 1, the PCUs are connected to the network 14 by wireless connections 22. Each PCU 12 has a tag reader 50 and a display 52.

The RABA server 16 maintains a database 24, in which the server 16 logs information about encounters between the care provider and the patients as recorded by the PCUs 12. Because the PCUs 12 are coupled via wireless connections 22, the PCUs 12 may continue to communicate with the server 16 as the care provider moves throughout a care facility, or other location where care is to be given, to provide services to various patients.

As previously noted, an RFID tag 30 is located in each patient's room 32 or other specified location and is specifically identified with that location. The RABA system 10 automatically registers each tag location and associates it with a patient 34 who is in a predetermined proximity to that particular tag 30. When the care provider's PCU 12 comes within range of a particular RFID tag 30, the RABA system 10 identifies the patient associated with that tag and retrieves active case information for that patient from the database 24. This information is presented to the care provider on the PCU display 52.

Each PCU has an associated biometric scanning device 80. The device may be incorporated within the PCU or be a separate device in communication with the PCU as shown by the two PCUs shown in FIG. 1. According to a preferred embodiment, the biometric scanning device is a palm scanner that uses Vascular Infrared Verification (VIV) technology to scan an image of a person's subcutaneous vascular pattern. This method of biometric authentication has been shown experimentally to have an accuracy of 99.996%. Each person's unique vascular pattern is matched to a database record created by pre-enrolling at least one hand of each patient and at least one hand of each care provider with the palm scanner device. It is preferred to have both hands of each patient and care provider pre-enrolled and placed in the database record so that biometric authentication may be performed during those times when one hand of the patient or the care provider may be temporarily unavailable, such as in a cast. Alternatively, other biometric scanning devices could be used such as retinal scanners, fingerprint scanners, etc., as would be understood by persons skilled in the art. However, the palm scanner is preferred since it presently provides greater accuracy, reliability and ease of use as compared with alternative devices.

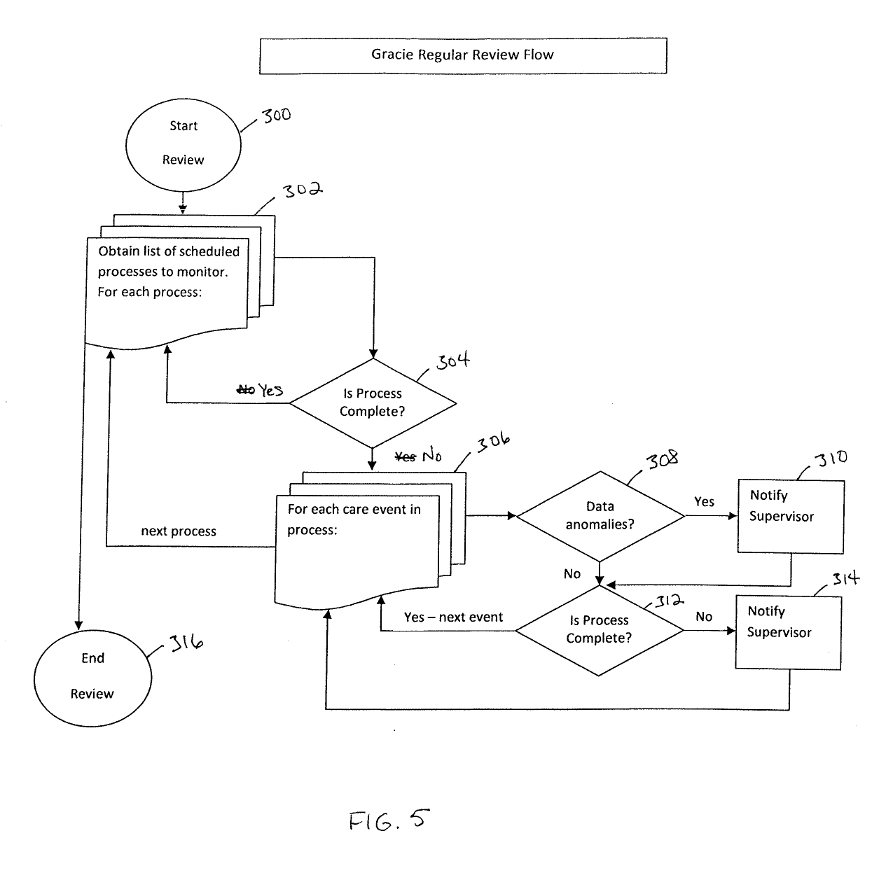

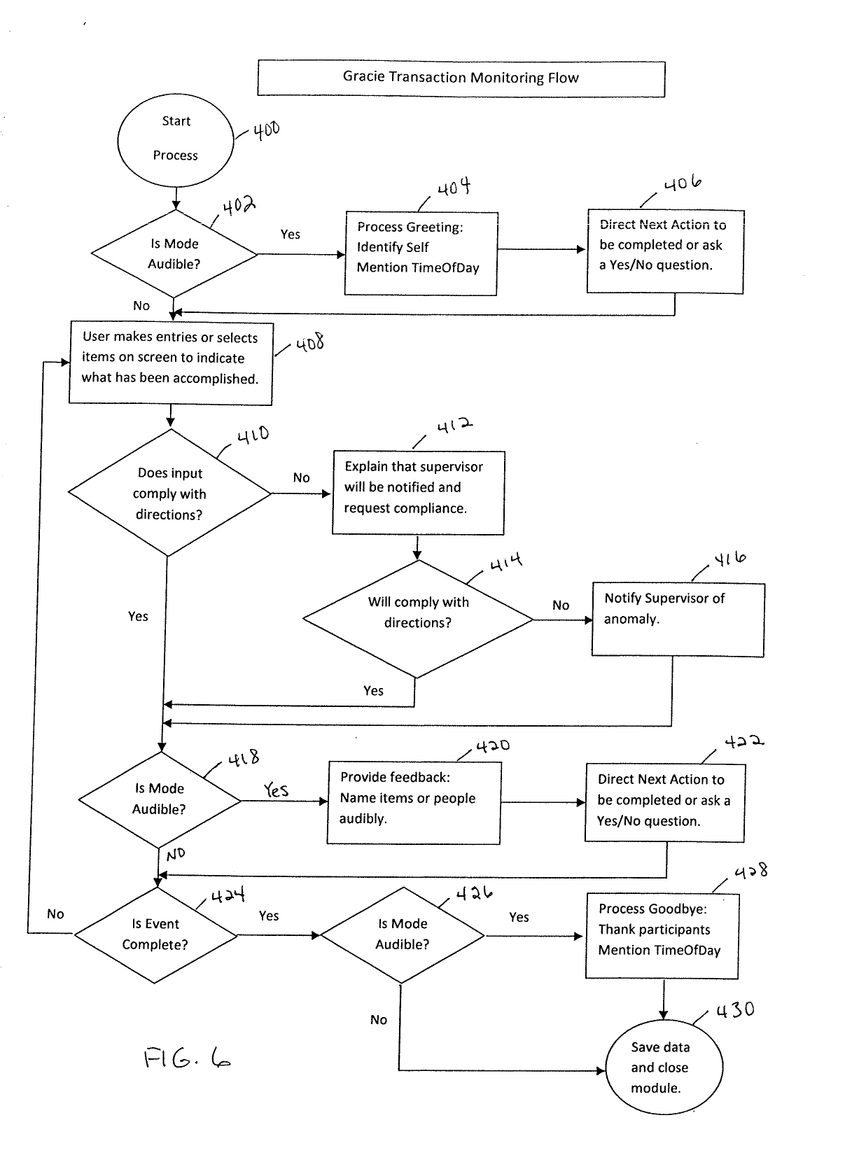

Each PCU 12 also has an electronic voice module (EVM) 82 that generates a virtual persona (VP) as a vocal participant and supervisor of all the interactions between the care provider and the patient who is receiving the medications and/or services. The virtual persona, which is preferably part of the system computer, provides auditory and/or visual confirmation of the identities of the patient and care provider which serves as a secondary form of biometric authentication. The virtual persona also audibly and/or visually presents the patient with a description of the service that is to be performed. The visual presentation is set forth on the PCU touch screen to assist when one of the participants in the care event is hearing-impaired. The virtual persona as created by the electronic voice module 82 not only facilitates accurate identification of the parties and the services being provided, but also provides audible and/or visual verification of the patient's decisions pertaining to receipt or rejection of services as well as the repercussions of the patient's refusal of service(s). According to a preferred embodiment, the VP also provides a voice recognition capability to be used by persons unable to manually access the system to provide responses. The virtual persona contains artificial intelligence algorithms to review transactions as they are collected and will notify supervisors of anomalous events. Flow charts (FIGS. 5 and 6) and further description of the virtual persona are provided hereinafter.

In addition to storing biometric identification data for each patient and care provider, the database 24 also serves as a repository of a range of other information. The database 24 is compiled from four primary sources, the Medical Administration Record (MAR) 60, the Plan of Care (POC) 62, the patient's Face Sheet 64, and the patient's billing information. The physician's diagnosis, prescriptions, and instructions are communicated electronically to the RABA system server 16 and recorded in the patient/resident's MAR 60 on the database 24. The POC 62, which is derived from the physician's instructions by properly trained personnel, provides the basis for service delivery. Personal and financial information is included in the patient's Face Sheet 64.

To manipulate the data, the RABA system has four primary software modules that are configured to utilize the data contained in the patient's MAR, POC, financial information, and Face Sheet stored in database 24. These modules are the Medication Management Module (MMM) 70, the Resident Services Module (RSM) 72, the Dietary Module (DM) 74 and the Financial Module (FM) 76.

The Medication Management Module 70 uses the information in the patient/resident's MAR 60 to electronically manage and record all aspects of prescription filling, drug inventory, medication dispensing and consumption, as well as electronic prescription reordering.

The Resident Services Module 72 utilizes instructions within the patient/resident's POC 62 to manage and account for services rendered to the patient. These services may include, but are not limited to, reporting of vital signs, blood sugar tests and insulin injections, and the use of inhalers and nebulizers. In addition, the Resident Services Module 72 also tracks the administration of services relating to basic care including, but not limited to, bathing, toiletry and transfer of the patient between resting locations such as from a chair to the bed.

The Dietary Module 74 provides a coordinated menu of dietary choices based on the POC 62 ordered by the patient's physician. The Dietary Module also records the patient's menu selection, and the location(s) of meal service and consumption.

The Financial Module 76 provides an accounting of all financial transactions between the patient and the facility where the patient is a resident. The Financial Module is used for assessing patient financial qualifications for admission, billing for services, and dispensing of resident funds. The Financial Module also serves as a tool to assist the patient in managing his or her finances.

Another aspect of the present invention pertains to providing notification(s) to one or more supervisors or other remote/interested parties 86 in real time upon the occurrence of certain events or anomalies. Anomalies can include, but are not limited to, the patient's refusal of, or unavailability for, a service; a test result outside a predetermined range of “acceptable” results for that test; a lack of correspondence between a medication as scanned during a care event for administration to a patient and the list of medications indicated for that patient, etc. As used herein, “supervisor” or “supervisors” is understood to include other third parties located either remotely or on site who have an interest in monitoring the RABA system and the patients being served.

Upon the occurrence of an anomaly, the transaction is put on hold. Supervisors must review the anomalies and take appropriate action before the care event can resume and the transaction can be completed. While the anomalous action is put on hold, for efficiency the care providers are permitted to continue on to the next task while supervisors are dealing with the anomaly. For example, if the patient refuses to accept service, the care provider enters the patient's refusal into the PCU 12. This entry triggers electronic generation and transmission of a notification message to a supervisor 86. Similarly, when the patient has accepted the service and the results obtained are outside an acceptable range, the PCU triggers electronic generation and transmission of a notification to the supervisor 86.

In addition, a supervisor 86 may wish to interact or communicate with a care provider PCU 12 or the server 16. The supervisor 86 is shown in FIG. 1 as being coupled to the wireless network 14. The supervisor is thus able to communicate in a wireless manner either with the care provider PCUs or with the server 16 via the wireless network 14 (or some other wireless network) that couples to the network 14. The supervisor 86 may also be coupled to the network 40 as shown in FIG. 2.

A supervisor 86 may also be interested in monitoring or receiving patient care status information. In one embodiment, the supervisor can interact with the server 16 to access status information pertaining to the patients having their care managed and documented by the RABA system. In another embodiment, the supervisor 86 can interact with the server 16 to configure type and/or frequency of conditions that are to cause the care provider PCUs to issue a notification to the supervisor 86. When these conditions are met, the system generates and sends a “system alert” to the supervisor, notifying him or her that something has happened which the supervisor had previously identified to the system as a notification event.

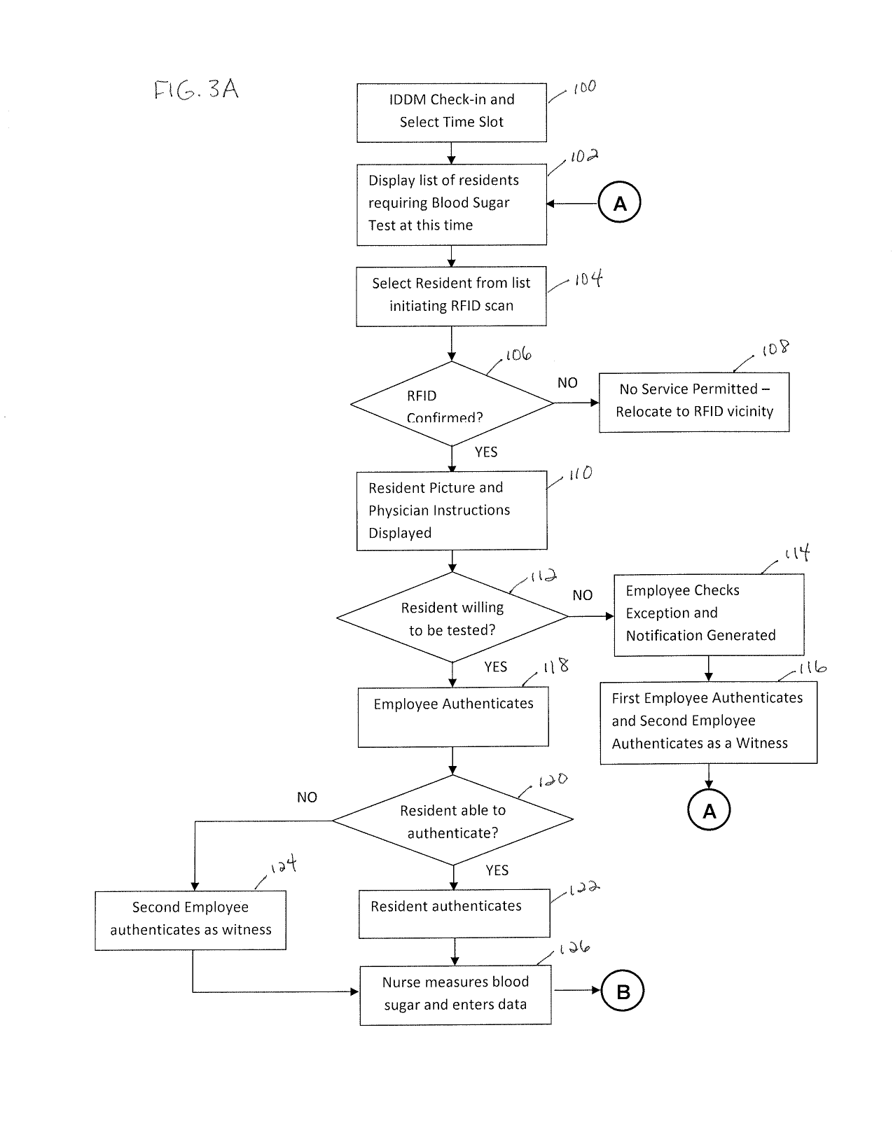

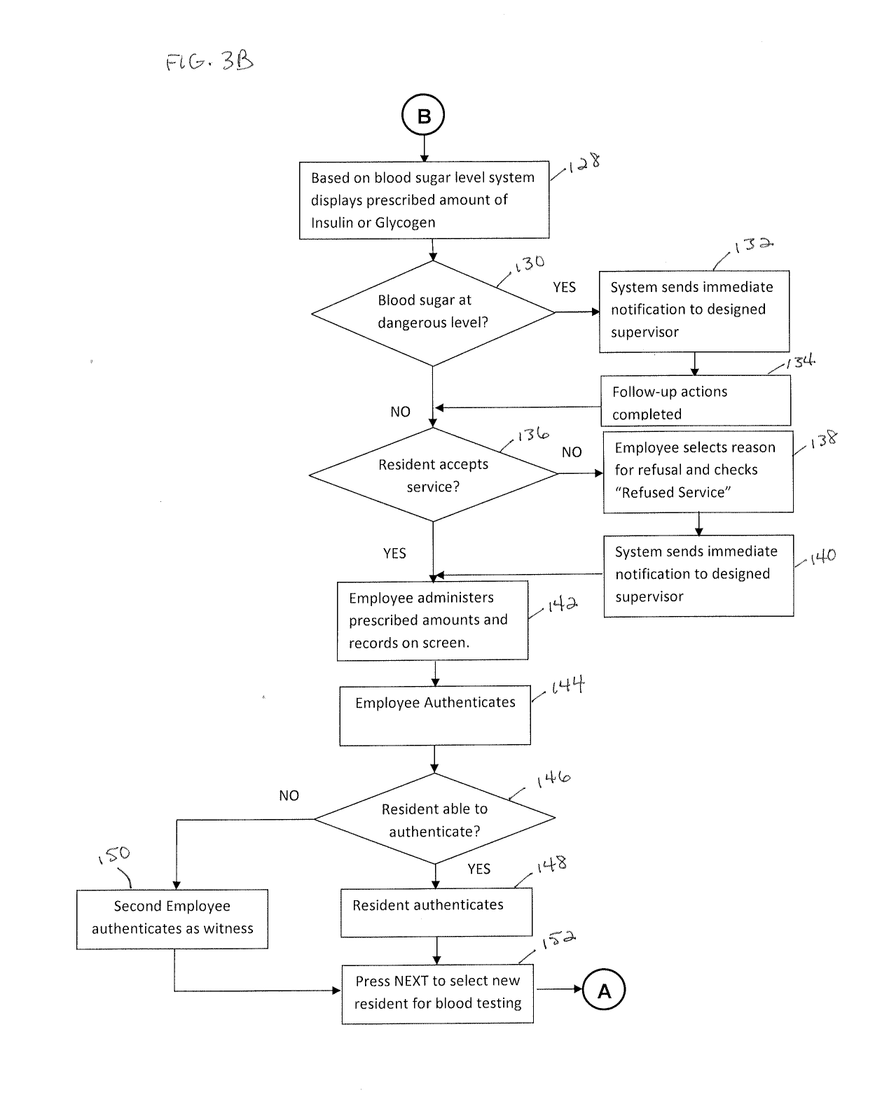

FIGS. 3A and 3B are a flow chart illustrating an example of the method using the RABA system according to the present invention. For the purposes of description, the flow chart is directed to the specific care event of blood sugar measurement using the Insulin-Dependent Mellitus (IDDM) component of the RSM 72. However, this is only an example as many other care events are intended to be performed and tracked using the RABA system 10.

As is known in the medical field, blood sugar tests are performed at regular intervals and/or particular times. The care provider begins the IDDM component of the RSM 72 by checking in and selecting a time slot, step 100. An anomaly, an action occurring outside the scheduled time frame, or non-compliance by the participants with the plan of care will result in a system-generated, real-time notification to a supervisor for further action as previously described.

Based on the time slot selected, the RABA system server 16 will retrieve and display a list of patients requiring blood sugar testing at that time, step 102. The care provider selects a patient from the list and then enters the patient's room with the PCU 12 to initiate the RFID scan, step 104. The RFID reader on the PCU communicates, either actively or passively, with the RFID tag at the patient's location, step 106. A successful RFID signal communication verifies the physical proximity of the care provider and the patient at the associated tag location. If proximity is not confirmed, step 106, no service is permitted, step 108, and the care provider returns to the list of patients, step 102.

Once proximity has been confirmed, step 106, the system electronically retrieves the patient information needed for the services that are scheduled to be provided. For example, the patient/resident's name, photo and the list of scheduled medications and services are retrieved from the MAR and POC schema and are displayed on the PCU for review by the care provider, step 110. The care provider enters information indicating whether the patient is present and willing to be tested, step 112. If the patient is not present or is unwilling to be tested, the care provider enters the patient's unavailability or refusal and the PCU electronically generates and sends a real-time notification signal to a supervisor, step 114. The care provider then uses the biometric scanner to authenticate his/her identity and, for verification purposes, a second care provider or other employee, acting as a witness, authenticates his/her identity, step 116. The biometric authentication by two persons present for the care event serves to verify and document that the service was not provided. According to a preferred embodiment, prior to the second biometric authentication, the PCU communicates with the RFID tag a second time to verify that the two care providers are still in proximity to the associated tag location. The care provider then returns to the list of patients requiring blood sugar testing at that time, step 102, in order to select another patient.

If the patient is present and willing to be tested, step 112, the care provider authenticates his/her identity using the biometric scanner, step 118. If the patient is able to authenticate, step 120, the patient then authenticates his/her identity using the biometric scanner, step 122. If the patient is not able to authenticate, a second care provider or other employee, acting as a witness, authenticates the second care provider's identity, step 124. As described in more detail hereinafter, if activated, the VP audibly and/or visually confirms the identities of the parties and also describes the service to be performed. Following all necessary identity authentications, the care event can be performed and, in this example, the care provider measures the patient's blood sugar and enters the results in the PCU, step 126.

Based on the blood sugar level, the RABA system displays the prescribed amounts of insulin or glycogen, step 128. If the blood sugar is at a dangerous level, step 130, the system sends an immediate notification to designated supervisory personnel, step 132. The supervisory personnel then take appropriate follow-up actions, step 134.

If the blood sugar is not at a dangerous level, step 130, the care provider determines whether the patient will accept the services, step 136. If the patient refuses service, the care provider enters an explanation of the refusal into the PCU, step 138. As will be described more hereinafter, the VP, if activated, will audibly and/or visually advise the patient that the patient's refusal will be reported to supervisory personnel. If the patient continues to refuse service, the system sends an immediate notification to designated supervisory personnel, step 140.

If the patient does accept the services, step 136, the care provider administers the prescribed amounts of insulin or glycogen and records these amounts and their administration using the touch screen on the PCU display, step 142. Following administration of the services or, in the event of patient refusal and the sending of a notification, the care provider biometrically authenticates his/her identity a second time, step 144. If the patient is able to authenticate, step 146, the patient then authenticates his/her identity a second time using the biometric scanner, step 148. If the patient is not able to authenticate, the second care provider or other employee, acting as a witness, authenticates the second care provider's identity, step 150. The requirements for a repeated biometric authentication of at least the patient and one care provider, or of two care providers, participating in the care event at the conclusion of the event provides an unbroken chain of accountability from the start to the completion of a transaction.

Also, according to a preferred embodiment, the physical proximity of the care provider and patient, or of the two care providers, to the RFID tag is also verified a second time prior to the second biometric authentication. The second RFID tag verification provides evidence that the care provider and patient, or the two care providers, are still in the appropriate location upon completion of the care event, or attempted care event in the event of patient refusal. Following all conclusory identity and proximity authentications, the service transaction is complete and the care provider, through appropriate entry to the PCU, returns to the patient list to select another patient, step 152.

According to the RABA system, the above-described steps are performed in linear sequence. Further care events cannot be conducted until both the care provider and the patient or the care provider and a witness have biometrically authenticated their identities at the completion of the previous event. While the IDDM component of the RSM has been summarized in FIGS. 3A and 3B, the sequence of steps undertaken, including RFID proximity verification, identity authentications before and after the service, second RFID proximity verification after the service, and issuance of appropriate notifications would be followed in the same manner when completing other care components within the RSM.

There may also be additional steps undertaken during a care event depending upon the specific module and care component. For example, the Medication Management Module (MMM) has a medication dispensing module, referred to herein as the MedPass Module, that includes steps to provide additional security and accountability in connection with the dispensing of medication.

The MedPass Module is used at a care facility to distribute specific dosages of medication to patients at scheduled times and locations. As with the other modules, the MedPass Module manages a proprietary sequence of biometrically authenticated transactions that (1) begins with a proximity-based RFID tag verification that triggers the beginning of a time period within which initial biometric authentication of the participants must be completed, (2) followed by a linear, sequence of managed care events, and (3) closed with a final proximity-based RFID tag verification which triggers a final biometric confirmation. In each of the two biometric actions, at least two parties must be successfully authenticated, the employee or care provider providing the care or service, and the patient or resident receiving the care or service. If the patient/resident is unable or unwilling to biometrically authenticate, then a second employee, acting as a witness, must biometrically authenticate in place of the resident.

Within the MedPass Module in particular, additional steps are taken to ensure accountability with respect to the medications being dispensed. Rather than just one care provider, two care providers or attendants, a lead and an assistant, use specialized, proprietary medication delivery carts (referred to herein as MedCarts) or, alternatively, hand-held devices (HHDs) to deliver the medications. The MedCarts and HHDs are inventoried within a medication storage area referred to herein as a MedRoom. Each of the MedCart and the HHD is a particular type of PCU.

Whether the MedCart or a HHD is used, each contains peripheral devices required by the process, as previously described, to record the location of the care event, which care providers and patients are involved in the care event, the beginning and ending times of the care event, and the services and/or care items provided during the care event. These peripheral devices typically include an RFID reader, a computer with a touch-screen, a barcode scanner and a biometric reader (palm or fingerprint). In addition, two sealed “rejection” bins are also provided to the attendants. These rejection bins are used to return any rejected medications, the first rejection bin being for controlled substances and the second rejection bin being for regular medications.

Medications scheduled to be provided to each patient are set-up in specialized dispensing bins by a prior process not described herein. The medications for a specific time period are segregated in barcoded dosage packets and placed in a corresponding barcoded dispensing bin. These dispensing bins are then either collected into trays arranged by distribution location, such as one floor of a building, so a MedCart will be able to go to an area of the care facility and service each patient location in that area, or the bin is placed into a HHD to be distributed to a specific resident. The MedCart and the HHD are also each provided with two rejection bins as previously identified.

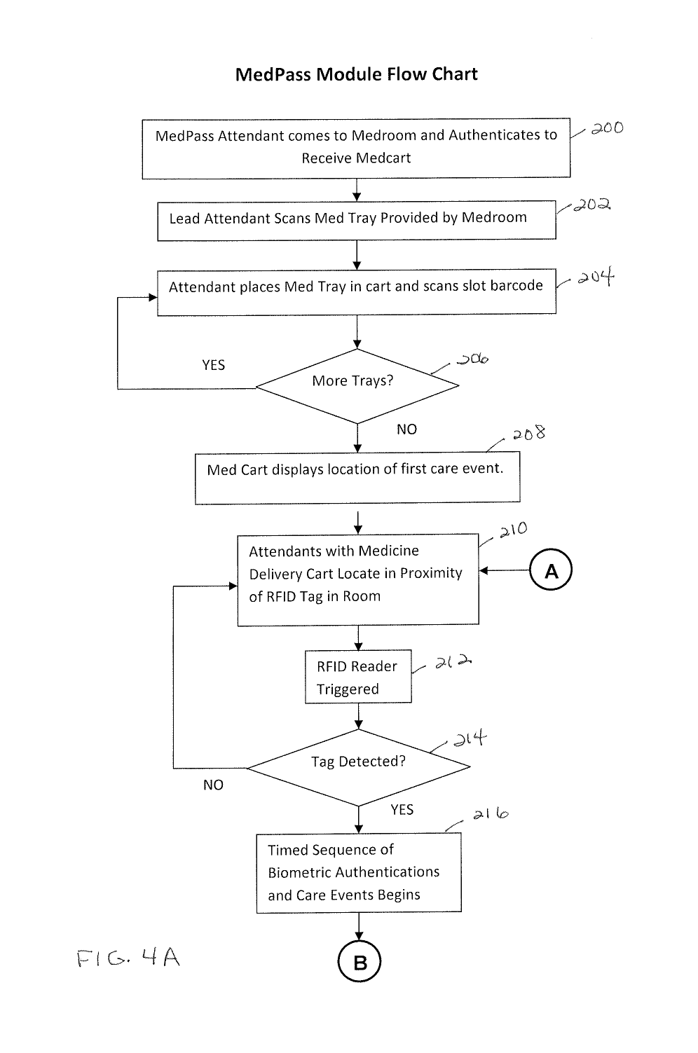

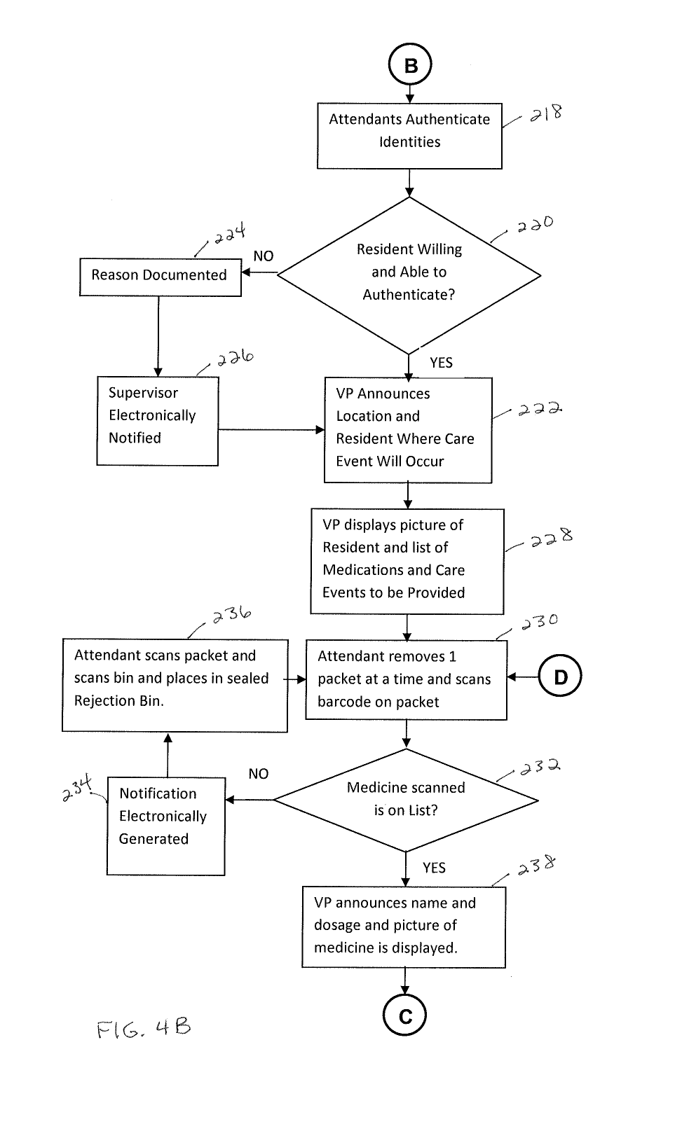

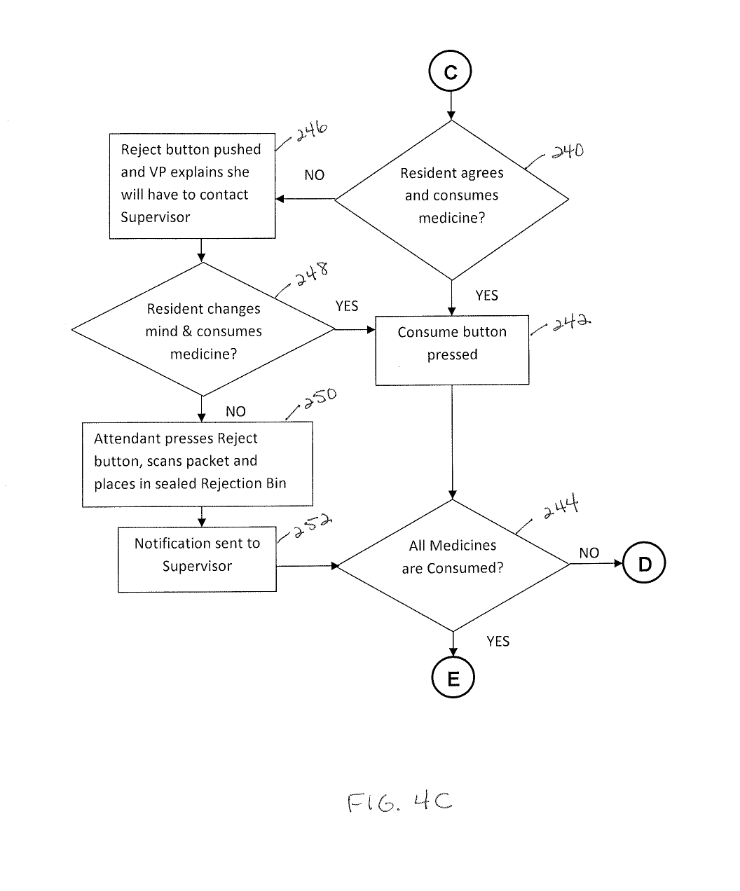

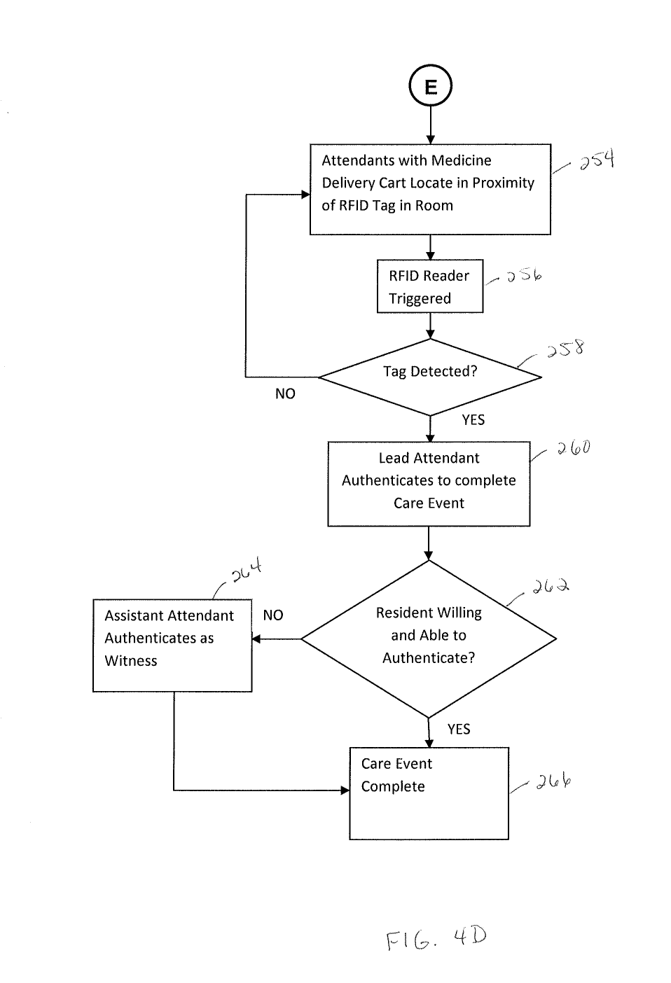

FIGS. 4A, 4B, 4C and 4D set forth a flow chart summarizing the steps undertaken when the care event is a medication dispensing service using the MedPass Module.

Two attendants of the MedRoom initiate the MedPass process by taking possession of a MedCart or a HHD and biometrically authenticating themselves on either the MedCart or HHD, step 200. If the MedPass will be completed using the MedCart, the attendants take possession of one or more trays containing the appropriate bins of medication from the MedRoom by scanning the barcode on each tray, step 202. The attendants then slide the tray into one of the empty slots in the cart, and scan the barcode associated with that slot on the MedCart, step 204. If there are more trays to be loaded, step 206, the attendants slide each tray into one of the empty slots and scan the barcode of the particular slot in which the tray was placed, step 204.

When all of the trays have been received, the MedCart or the HHD being used will direct the attendants as appropriate. If the MedPass will be conducted with a HHD, the HHD computer will direct the attendants to a specific location. If the MedPass will be conducted with a MedCart, the MedCart computer will direct the attendants to the first location on its scheduled trip, step 208.

When the attendants arrive at the designated location, they locate the MedCart or HHD in proximity to the RFID tag at that location, step 210. The lead attendant will then trigger the RFID reader to detect the RFID tag which must be at that location, step 212. If the appropriate tag is not detected, step 214, the attendants reposition the MedCart or HHD so that the tag can be detected.

If the appropriate tag is detected, step 214, additional information about the event is electronically retrieved from the server and a timed sequence of scheduled biometric and electronically verified events begins, step 216. Both of the attendants and the patient scheduled to receive the care must be biometrically identified, step 218, within a specified time period following RFID tag detection, step 214. Failure to complete the biometric authentications within the time period results in suspension of the care event and transmission of an electronic notification to the supervisor. If the patient/resident is not willing and able to authenticate, step 220, then a reason must be provided and one of the attendants enters the reason for non-compliance into the MedCart of HHD computer, step 224. The system then electronically notifies a supervisor of the anomaly, step 226.

If the patient/resident is willing and able to authenticate, step 220, and a VP is activated to communicate, the VP audibly announces and/or visually displays the room location and the name of the person receiving the care or service, step 222. For ease of description, the VP in audible mode is assumed to be a woman's voice and therefore is referred to in the female gender. As would be understood, the VP may be set up with a male voice and in any language.

As each participant is biometrically identified, their picture is displayed on the MedCart or HHD, step 228, and the VP audibly announces and/or visually displays the name of each person. After the participants have been confirmed, a list of the medicines prescribed by the patient's physician to be taken at that time and any instructions from the physician associated with this medicine are audibly announced by the VP and/or displayed on the touch screen, step 228.

The lead attendant then removes one dosage packet at a time from the appropriate dispensing bin set up for the patient and scans the barcode on the packet, step 230. The computer will determine if this barcode is assigned to one of the medications on the list for that patient at that time, step 232. If the medication is not on the list or is not scheduled to be provided to that patient/resident at that time, a supervisor is electronically notified of the anomalous event, step 234, and the lead attendant is directed to scan the appropriate rejected medications bin and place the medication into that bin for return to the medication inventory, step 236.

If the medication is on the list, step 232, the MedCart or HHD computer will display a picture of the medication at the proper dosage and the VP will audibly announce the name and dosage strength of the medication, step 238. If the resident/patient agrees to consume the medication, step 240, the assistant attendant will open the packet and provide the dosage to the patient with water. Once the medicine has been consumed, the lead attendant will enter an input to the MedCart or HHD computer, such as by pressing a touch screen field or button designated “Consume” to signify and record that the medication was consumed, step 242.

If the resident refuses or is unable to take the medication, step 240, the lead attendant will input this refusal, such as by pressing a touch screen field or button designated “Reject”. The VP will then audibly explain, or provide a warning on the touch screen, that she will have to contact the supervisor about the patient's refusal, step 246. If the resident then decides to take the medication after all, step 248, the attendant will provide the patient with the medication and, once it is taken, press the Consume button, step 242. However, if the resident still refuses to take the medication, the attendant enters the reason for the rejection into the computer, step 250. Then the lead attendant will put the medicine back into the packet and reseal it. All rejected medicine packets are rescanned, the appropriate sealed rejection bin (normal or controlled) is scanned, and the medicine is placed into the bin, step 250. A supervisor is also electronically notified of the anomoly, step 252.

The MedCart or HHD computer will then redisplay the remaining list of medications and associated instructions; if there are more medications to be dispensed, step 244, the above sequence of steps, beginning with step 230, will be repeated for each medication to be provided. Once all medications have been provided that are in the list of scheduled medications for a particular care event, step 244, the attendants again locate the MedCart in proximity to the RFID tag at the care event location, step 254. The RFID tag reader is triggered again to validate that the PCU is still in the required location, step 256. If the tag is not detected, step 258, the attendants move the MedCart as needed to be in proximity to the RFID tag, step 254. Once the tag is detected, step 258, the lead attendant and the patient/resident must each biometrically authenticate themselves to confirm that the scheduled sequence of care events was completed, step 260. If the patient is unwilling or unable to biometrically authenticate themselves, step 262, then the assistant attendant must biometrically identify himself or herself as a witness, step 264. If the resident is willing and able to authenticate, then the resident authenticates, step 262. The VP, if activated, will audibly and/or visually thank the patient and say goodbye. The care event is then complete and the MedCart is returned to the MedRoom, step 266. This concludes the transaction.

If the MedPass is being conducted with a MedCart, the two attendants will be directed to the location of the next scheduled care event. Once all care events at all scheduled locations are completed, the two attendants will return the MedCart or HHD to the Medroom.

At the Medroom, the two attendants will logout of the unit by biometrically authenticating themselves, and then turn the MedCart/HHD over to the MedRoom. An attendant of the MedRoom must also biometrically authenticate himself or herself to accept the unit. Once the unit is back in the MedRoom, the MedPass process is complete. Later, in a separate process not described herein, an inventory manager will remove the medications from the rejected medication bins and return the medications to inventory.

When completing a care event within both the MedPass Module and the IDDM component of the RSM, the VP regularly reviews system activities to determine if all scheduled events have been completed properly and on time, and also reviews transactions (care events) as they are in process.

The regular review function of the VP is summarized in FIG. 5. At various scheduled times, the VP will review all monitored processes to ensure that each care event has been completed properly and will electronically notify the appropriate supervisory staff of any overdue or anomalous events. As part of this review the VP has the task of creating data that will be used in later events. For example, the VP creates the data records for IDDM entries scheduled for each time slot for the coming day based on entries in the patient's MAR.

The regular review process begins, step 300, with the VP obtaining a list of scheduled processes to monitor, step 302. For each process, the VP determines if the process is complete, step 304. If the process is complete, the VP returns to step 302. If the process is not complete, the VP sequentially reviews one or more care events that are still in process, step 306. For each care event, the VP checks for data anomalies, step 308, and, if anomalies are found, the VP notifies a supervisor, step 310.

The VP will contact the supervisor in the method chosen by the supervisor as their preferred contact method, which is defined for each type of care event. Methods that may be used include text message to a phone, email to a phone or computer, or “system alert”. The “system alert” may be sent to what is known as the system “TO DO” box which contains a list of items that each employee is scheduled to complete. The list typically contains action items having an associated date and time at which the action is to be taken, and also contains system alert items such as “Something happened that you want to know about”, as in data anomalies. Each notification contains a one-time auto-login hyperlink back to the system where the supervisor can review the anomalous data in order to make an appropriate decision about what needs to be done and if the care event can resume.

If there are no data anomalies, step 308, or if the supervisor has completed his or her review of the data anomalies, the VP determines whether the process is complete, step 312. If the process is not complete, indicating another problem, the VP notifies a supervisor, step 314. If the process is complete, step 312, the VP moves to the next care event still in process, step 306. Once all care events still in process have been reviewed, the VP returns to the list of scheduled processes, step 302. If there are no remaining scheduled processes, the review ends, step 316.

In the transaction monitoring role, the VP may or may not make herself known with audible and/or visually output. The VP will review each step of each transaction that is being monitored to ensure that it is within expected parameters. Any anomalous action will result in the VP notifying the appropriate supervisor. If the VP is activated to communicate in audible mode, she will comment about and direct the care event with audible statements, in addition to looking for anomalies. When in audible mode, the VP uses polite, kind, caring third person language to discuss the current care event and to direct the next action of the care providers and the patient receiving the service. In the case where the patient refuses or fails to comply with her directions, the voice of the VP will change to using more forceful first person language and will take the conflict away from the care provider by explaining that it is she (the VP) who will have to contact a supervisor about the problem.

The transaction monitoring function of the VP, summarized in FIG. 6, begins, step 400, with the VP determining whether the mode she is in is audible, step 402. If the mode is audible, the VP processes a greeting in which she identifies herself and, in some cases, also mentions the time of day, step 404. Preferably, the VP mentions the time of day on a random basis (not every time) to give the VP the semblance of sentience. The VP then directs the next action to be completed, or asks a yes/no question related to the care event being performed, step 406. For example, in the MedPass Module, the VP may ask “will you consume the medication?”. The care provider makes an appropriate entry or selects an on-screen field displayed on the PCU to indicate what has been accomplished, step 408. For example, if the patient agreed to consume the medication and, in fact, did so, the care provider will press the Consume button.

After the appropriate indication of the action accomplished has been input into the PCU, step 408, the VP checks whether the input complies with the directions provided to the care provider with respect to the action to be undertaken in the given care event, step 410. If the input indicates non-compliance, the VP will audibly explain that a supervisor will be notified unless the patient consents to and allows the care event, step 412. If the patient does not comply with the directions provided, step 414, the VP notifies a supervisor of the anomaly, step 416.

If the patient does comply with the directions, step 414, the VP again checks to see whether the mode she is in is audible, step 418. If the mode is audible, the VP provides audible feedback including an announcement of the items pertaining to the care event and/or the persons participating in the care event, step 420. The VP then directs the next action to be completed, or asks a yes/no question related to the care event being performed, step 422. As an example of a concluding yes/no question, when operating within the Finance Module 76 the VP may ask, “do you want a printed receipt for the transaction?”. If the event is not complete, step 424, the care provider provides an appropriate input to the PCU, step 408, and the VP then repeats process steps 410 to 422.

If the event is complete, step 424, the VP checks to see whether the mode she is in is audible, step 426. If the mode is audible, the VP processes a concluding statement in which she tells the patient goodbye, thanks the participants and, in some cases, mentions the time of day, step 428. The VP then saves the data and closes the module, step 430.

As set forth herein, the present invention serves to enable monitored delivery of all medications and personal care services, to establish menus necessary to meet the patient's dietary needs, and to track and document all financial transactions. The present invention provides a system and method for performing all of these functions securely with the needed accountability to reduce the risk of fraud, waste and human error, while improving overall patient care. The requirement for two biometric authentications, one before and one after the care event, and both by authorized persons, ensures continuity of the proper participants throughout the care event and avoids blame casting and fraudulent practices that can arise when there are difficulties in completing the care event. Verification of proximity using RFID tags both before and after the care event provides evidence that the care event was, in fact, completed in the appropriate location. The inclusion of the VP, acting as a supervising party, further smooths over any conflicts between the patient and the care provider during the care event. The VP also clarifies the service(s) being performed to avoid errors by the care provider and confusion on the part of the patient.

The foregoing descriptions and drawings should be considered as illustrative only of the principles of the invention. The invention may be configured in a variety of shapes and sizes and is not limited by the dimensions of the preferred embodiment. Numerous applications of the present invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

What is claimed is:

1. A method for managing and tracking medical care and medical service transactions between a patient at a location identified by an RFID tag and a care provider having a portable care unit (PCU) with an RFID reader using a network connected application on a server coupled to a database with stored data pertaining to the patient, comprising:
confirming that the care provider PCU and the patient location are in proximity to one another based on communication between the RFID reader and the tag;
if proximity is confirmed, said PCU electronically retrieving information needed for a service to be performed from said database and displaying said information to the care provider on said PCU;
biometrically authenticating the patient's identity a first time using a biometric scanning device associated with said PCU or, if the patent is unable to authenticate, biometrically authenticating a second employee to act as a witness;
biometrically authenticating the care provider's identity a first time using a biometric scanning device associated with said PCU;
upon successful authentication of both patient and care provider identities, and following confirmation of the proximity of the PCU and the RFID tag, offering said service to said patient;
upon patient acceptance of said service, said care provider performing said service;
upon completion of said service, biometrically authenticating the patient's identity a second time or, if the patent is unable to authenticate, biometrically authenticating the second employee to act as a witness; and
biometrically authenticating the care provider's identity a second time to complete said service transaction.

2. The method as set forth in claim 1, further comprising the step of confirming, after the service has been performed and before the second biometric authentications, the proximity of the care provider and patient a second time using the RFID reader and tag.

3. The method as set forth in claim 1, wherein if proximity is not confirmed, no services are permitted to occur.

4. The method as set forth in claim 1, wherein said steps of biometrically authenticating include using a palm scanner as the biometric scanning device.

5. The method as set forth in claim 1, wherein said step of performing said service includes recording results using the PCU, determining whether results are in an acceptable range and, if said results are outside the acceptable range, said PCU electronically generating and sending a notification message to a supervisor over the computer network.

6. The method as set forth in claim 1, wherein if the patient refuses to accept service, said step of offering includes entering the patient's refusal into the PCU, said entry triggering electronic generation and transmission of a notification message from the PCU to a supervisor over the computer network.

7. The method as set forth in claim 1, wherein said steps of biometrically authenticating the patient's identity include biometrically authenticating the identity of two care providers even when the patient is able to authenticate.

8. The method as set forth in claim 1, further comprising, after the step of authenticating the patient's identity the first time, the step of said PCU activating a virtual persona as a vocal participant in the transaction, said virtual persona audibilizing the patient's identity for verification thereof.

9. The method as set forth in claim 8, wherein said step of offering said service includes having the virtual persona audibly present the patient with a description of the service to be performed.

10. The method as set forth in claim 8, wherein if said patient refuses to accept service, said step of offering includes having the virtual persona audibly acknowledge the patient's refusal and audibly notify the patient of non-compliance with patient's plan of care.

11. The method as set forth in claim 10, wherein upon said patient's refusal, the care provider enters the refusal into the PCU and the PCU electronically generates and transmits a notification signal to a supervisor over the computer network.

12. The method as set forth in claim 11, wherein upon said patient's refusal, the service transaction is suspended until supervisory review and/or action is complete.

13. A system for controlling and tracking transactions between a patient at a location and a care provider over a computer network, comprising:
a network connected application on a server connected to said computer network;
a database coupled to said server and storing a range of patient data in a plurality of database files by category;
an RFID tag at the patient location;
a portable care unit in communication with said network connected application over the computer network, said portable care unit having a touch screen display and an RFID reader configured to communicate with said RFID tag to verify proximity of the portable care unit to the patient location;
a biometric scanning device associated with said portable care unit, said scanning device configured to biometrically verify the identities of the patient and one or more care providers both before and after a given transaction;
said network connected application including a plurality of software modules, each of said modules configured to manipulate data from at least one of said database files to generate a patient course of action in one of said categories.

14. The system as set forth in claim 13, wherein said portable care unit includes an electronic voice module configured to create a virtual persona, said virtual persona configured to audibly confirm said patient and care provider identities following biometric authentication.

15. The system as set forth in claim 13, wherein said system is configured to electronically generate and send a notification signal to a supervisor upon occurrence of a specified event.

16. The system as set forth in claim 13, wherein said biometric scanning device is a palm scanner using VIV technology.

17. The system as set forth in claim 13, wherein said modules include a Medication Management Module configured to use patient data relating to a patient's medical administration record to electronically manage and record aspects of prescription filling, drug inventory, medication dispensing and consumption, as well as electronic prescription reordering.

18. The system as set forth in claim 13, wherein said modules include a Resident Services Module configured to utilize data relating to a patient's plan of care to manage and account for services rendered to the patient, such services including, but not limited to, reporting of vital signs, blood sugar tests and insulin injections, and the use of inhalers and nebulizers.

19. The system as set forth in claim 13, wherein said modules include a Dietary Module configured to provide a coordinated menu of dietary choices based on the plan of care ordered by the patient's physician, and to record the patient's menu selection, and the location(s) of meal service and consumption.

20. The system as set forth in claim 13, wherein said modules include a Financial Module configured to provide an accounting of all financial transactions between the patient and a facility where the patient is a resident.
